(19) United States
(12) Patent Application Publication
(10) Pub. No.: US 2006/0015032 A1
(43) Pub. Date: Jan. 19, 2006

(54) Non-invasive method for measuring changes in vascular reactivity

(76) Inventor: Linda Gordon, Pittsburgh, PA (US)

Correspondence Address: Alan G. Towner;Pietragallo, Bosick & Gordon, One Oxford Centre, 38th Floor, 301 Grant Street, Pittsburgh, PA, 15219, US

(21) Appl. No.: 11/181,108

(22) Filed: Jul. 14, 2005

Related U.S. Application Data
(60) Provisional application No. 60/587,808, filed on Jul. 14, 2004.

(51) Int. Cl. A61B 5/02

(52) U.S. Cl. 600/483; 600/485

(57) ABSTRACT

A method is disclosed for measuring changes in vascular reactivity that seeks to overcome the aforementioned shortcomings. The method is non-invasive and allows for continuous or sustained monitoring of blood vessels. By measuring changes in vascular reactivity, information on endothelium-dependent vasorelaxation may be provided. Parameters such as skin temperature, heat flux, ambient temperature, movement, galvanic response and/or body acceleration may be monitored to indicate changes in vascular reactivity and to provide information concerning endothelical function of patients. Other information such as patient response to blood pressure medications and other types of medications, onset of congestive heart failure and other types of medical complications, and the occurrence of unstable angina may also be obtained.

### CROSS-REFERENCE TO RELATED APPLICATIONS

This application claims the benefit of U.S. Provisional Patent Application Ser. No. 60/587,808 filed Jul. 14, 2004, which is incorporated herein by reference.

### FIELD OF THE INVENTION

The present invention relates to the measurement of vascular reactivity, and more particularly relates to a non-invasive method for measuring changes in vascular reactivity by monitoring certain parameters.

### BACKGROUND INFORMATION

Heart disease is one of the leading killers of adults in the United States. Fortunately, deaths due to heart disease have been declining in the last several decades, which is due in part to improved treatments and better efforts to modify risk factors. Much of this progress is based on a more complete understanding of the factors that can lead to heart attacks.

Heart attacks, known medically as myocardial infarctions, occur when some portion of the heart muscle dies due to lack of blood flow. Blood flow is cut off when atherosclerotic plaque, made up in part of cholesterol deposits, ruptures and causes a blood clot to form. Because plaque develops on the lining of the coronary arteries, it affects the behavior of those blood vessels. They tend to remain tense when they should dilate, which interferes with the delivery of oxygenated blood to the heart.

This increased vascular tension of the coronary arteries is known as endothelial dysfunction, and this endothelial dysfunction has been shown to be one of the earliest signs of developing heart disease. By treating heart disease risk factors such as high blood pressure or high cholesterol, the endothelial function can be normalized. Restoration of normal endothelial function appears to correlate with a significant reduction in risk for heart attack.

One of the interesting things about endothelial dysfunction is that it appears to be a systemic phenomenon, meaning that it is similar throughout the body. This allows one to assess risk for heart disease by measuring the behavior of blood vessels, for instance, in the arm. A change in blood vessel behavior is known as vascular reactivity.

There are many known methods for measuring vascular reactivity. A major drawback to many of the known methods is that the procedures are invasive.

Intravascular ultrasound is a known invasive method for measuring vascular reactivity. Intravascular ultrasound involves inserting a catheter into an artery to view the cross section of the artery in real-time. A high-frequency transducer is used to enhance the resolution of the image and the accuracy of the measurement. An obvious drawback of this procedure is the invasiveness that is inherent with the use of a catheter. Another drawback of this system is that it can only be used with proximal arteries, because of the comparatively large size of the catheter. An additional problem is that the catheter itself can be a potential source of error, since it may interfere with blood flow.

Another catheter based approach for measuring vascular reactivity is Doppler velocimetry. Doppler velocimetry involves the use of a piezo-electric crystal mounted on the tip of catheter or on an extremely thin guide wire, which can be positioned in the artery of interest. Blood flow velocity is measured from the reflection of ultrasonic waves off of the erythrocytes in the blood vessel under study. Since Doppler velocimetry is an invasive catheter based approach, it suffers from the same drawbacks already noted above.

Angiography is a standard method for measuring vascular burden. Angiography involves the direct injection of a contrast agent into the blood vessel of interest to enable its radiographic visualization. By monitoring the contrast agent as it moves through the blood stream, changes in vascular dimensions can be measured. However, angiography only provides short glimpses of blood vessel measurements for no more than a few seconds, because the contrast agent moves quickly through the bloodstream. Thus, angiography is not suitable for continuous monitoring of vascular reactivity.

A non-invasive method involves the use of high-frequency ultrasound. This technique consists of bombarding the brachial artery from the surface with a 10-12 MHz transducer, and measuring the resulting diameter of the blood vessel. Flow-mediated endothelium-dependent vasodilation is then assessed by inflating a wrist cuff to supra-systolic pressures for a finite time period (5-10 minutes) and then releasing it, to allow a hyperemic response to occur in the forearm and hand. The brachial artery dilates in response to this hyperemia, and this dilation is endothelium dependent. A major limitation to this method is that it provides information only on flow-mediated vasodilation, which is only one aspect of endothelium-dependent vasorelaxation.

There is a need for a non-invasive method for measuring vascular reactivity that can provide continuous monitoring of the blood vessels. The method should also be able to provide accurate and consistent information on all aspects of endothelium-dependent vasorelaxation.

### SUMMARY OF THE INVENTION

The present invention provides a method for measuring changes in vascular reactivity that seeks to overcome the aforementioned shortcomings. The method is non-invasive and allows for continuous or sustained monitoring of blood vessels. By measuring changes in vascular reactivity, information on endothelium-dependent vasorelaxation may be provided. Parameters such as skin temperature, heat flux, ambient temperature, movement, galvanic response and/or body acceleration may be monitored to indicate changes in vascular reactivity and to provide information concerning endothelical function of patients. Other information such as patient response to blood pressure medications and other types of medications, onset of congestive heart failure and other types of medical complications, and the occurrence of unstable angina may also be obtained. For example, skin temperature and heat flux would decrease with congestive heart failure.

An aspect of the present invention is to provide a method of measuring vascular reactivity of a patient. The method comprises measuring a skin temperature and/or a heat flux from the patient, and measuring the vascular reactivity of the patient based on the measured skin temperature and/or heat flux.

Another aspect of the present invention is to provide a method of performing a stress test on a patient. The method comprises conducting the stress test, and measuring vascular reactivity of the patient by measuring skin temperature and/or heat flux from the patient during at least a portion of the stress test.

These and other objects of the present invention will be more apparent from the following description.

### DETAILED DESCRIPTION

The present invention provides a method for measuring changes in vascular reactivity. The method is non-invasive, is capable of providing sustained or continuous monitoring of the blood vessels, and is capable of providing information on aspects of endothelium-dependent vasorelaxation.

In one embodiment, vascular reactivity is measured by monitoring parameters that are sensitive to changes in vascular reactivity. These indicators may include, but are not limited to, skin temperature, heat flux, movement, heart rate, ambient temperature, galvanic response, and body acceleration. For example, skin temperature and heat flux may be measured in order to determine vascular reactivity of a patient. As used herein, the term “patient” includes any member of the animal kingdom including mammals such as humans.

The parameters may be monitored by any known monitoring device or sensor. In one embodiment, a monitoring armband may be used for measuring the parameters, such as but not limited to a monitoring armband sold under the designation SENSEWEAR PRO by BODYMEDIA. Although such a monitoring armband is worn on the back of the upper arm, sensors can be placed at other locations on the arm or body, such as the leg, chest, neck and/or head areas. It is to be understood that any other suitable type of monitoring device or sensor may be used in accordance with this embodiment.

The monitoring device or sensor may measure heat flux. The heat flux sensor measures the amount of heat being emitted by the body. The heat flux sensor may measure representative values of heat convection by air in contact with the skin which is part of the total thermal energy dissipated to the surroundings. The heat flux sensor may be placed in a thermally conductive path between the skin and the outer side of the armband or other device exposed to the environment. A high gain internal amplifier may be used to bring the temperature signal to a level that can be sampled by a microprocessor.

The monitoring device or sensor may measure skin temperature. Skin temperature may be measured with a thermistor-based sensor located on the back-side of the device in contact with the skin. As the skin temperature changes, a change in the electrical resistance of the sensor causes a change in the voltage sampled by the microprocessor.

In accordance with the present invention, temperature and/or heat flux measurements may be used to detect vascular reactivity such as vasodilation characteristics. Increased vasodilation results in more heat being given off by a patient's body and may also result in decreased body temperature. Patients with heart disease tend to have decreased abilities to vasodilate in comparison with healthy patients. This decreased capacity to vasodilate results in less heat being given off from the body of the patient and can be detected by the temperature measurement and/or heat flux detection method of the present invention.

Ambient temperature may also be measured. For example, a near body ambient temperature sensor may be attached to the heat flux sensor. Ambient temperature is affected by heat rushing up and off the body around the sensor and its interaction, with the environmental conditions. The rate of change in the ambient temperature can also be used to indicate the type of physical activity and to verify that the heat flux sensor is not receiving noisy signals.

Heart rate may also be measured. Several companies manufacture chest straps for heart beat detection. These devices transmit a 5 kHz burst of electromagnetic energy, which may be detected by the armband sensor having a receiver board to receive the pulses emitted by the heart detection chest straps. A high quality data stream that captures every beat can be analyzed to provide beat-to-beat variability.

In one embodiment, a non-invasive body temperature and/or heat flux monitor gathers data from a patient undergoing standard stress testing, such as adenosine stress testing, etc. The temperature and/or heat flux measurements may be used as an indication of blood pressure changes, e.g., before, during and/or after the stress testing.

In another embodiment, the present invention may be used for the purpose of assessing a patient's response to certain medications. Example medications may be blood pressure medications, but a patient's response to other medications may be assessed in accordance with this embodiment.

In a further embodiment, the present invention may be used for the purpose of predicting the onset of certain medical complications. An example medical complication may be congestive heart failure, but the onset of other medical complications may be predicted in accordance with this embodiment.

In another embodiment, the present invention may be used for the purpose of assessing unstable angina in a patient.

In one embodiment of the invention, a patient may be fitted with a monitoring armband, such as an armband sold under the designation SENSEWEAR PRO by BODYMEDIA, which is worn over the tricep area. The armband utilizes a combination of sensors that continuously gather data about movement, skin temperature, ambient temperature, heat flux, and galvanic skin response. The armband may be worn by the patient for, e.g., thirty (30) minutes in order to provide a baseline for the data collection. The patient may then be injected with a dose of adenosine. The adenosine can be injected into an intravenous site anywhere on the patient's body. For example, approximately 0.56 milligrams of adenosine per kilogram may be injected into the patient over a period of four (4) minutes. Adenosine, when injected, will cause the arteries to dilate. The time when the adenosine dose is injected is noted and recorded. After approximately ten (10) seconds, the adenosine begins to cause the patient's arteries to dilate. This time is also noted and recorded. The armband continues to collect data for, e.g., approximately thirty (30) minutes after the injection of the adenosine. A polar heart rate monitor may be used to simultaneously and wirelessly transmit heart rate data to the armband or another receiver. The collected data may be uploaded to a computer for analysis and evaluation. The data gathered will be uploaded to a software program and analyzed by BodyMedia InnerView Research Software. BodyMedia's InnerView Research Software is a Java-based software application that will enable us to adjust data collection channels and sensor sampling rates on the SenseWear Pro Armband as well as upload, analyze, profile, and trend physiologic data that the armband collects. We will then utilize standard statistical software to analyze this data.

Blood pressure may be monitored at the onset of the foregoing stress testing. Blood pressure may be recorded during the infusion of adenosine. While the blood pressure is recorded, the armband sensor may continuously record skin temperature, ambient temperature, heat flux, gravity, and/or motion. The change in systolic blood pressure may be correlated to the change in skin temperature. This method recognizes the relationship between skin temperature change and altered vascular tone, as measured by changes in systolic blood pressure, following adenosine administration.

Although not intending to be bound by any particular theory, there is a negative correlation between systolic and diastolic blood pressure changes (i.e., decrease) and heat flux and skin temperature changes (i.e., increase), for example, as measured during an adenosine stress test. A standard ANOVA (analysis of variance) may be used to analyze the data gathered to calibrate the extent to which the heat flux and skin temperature changes separate the blood pressure readings. Due to the relationship between brachial artery dilation and coronary artery disease, the non-invasive sensor provides a measure of coronary artery function.

Whereas particular embodiments of this invention have been described above for purposes of illustration, it will be evident to those skilled in the art that numerous variations of the details of the present invention may be made without departing from the invention as defined in the appended claims.

1. A method of measuring vascular reactivity of a patient, the method comprising:
measuring a skin temperature and/or a heat flux from the patient; and
measuring the vascular reactivity of the patient based on the measured skin temperature and/or heat flux.

2. The method of claim 1, wherein skin temperature and/or heat flux are measured by at least one sensor worn on the body of the patient.

3. The method of claim 2, wherein the sensor is worn on the arm of the patient.

4. The method of claim 2, wherein the sensor is provided in an armband.

5. The method of claim 2, wherein the sensor measures both of the skin temperature and heat flux.

6. The method of claim 5, further comprising measuring an ambient temperature near the body of the patient.

7. The method of claim 2, wherein the sensor is further capable of measuring movement, galvanic response and/or body acceleration of the patient.

8. The method of claim 1, further comprising measuring a heart rate of the patient.

9. The method of claim 1, wherein the measured vascular reactivity includes vasodilation.

10. The method of claim 1, wherein the measured vascular reactivity includes systolic blood pressure changes.

11. The method of claim 1, wherein the measured vascular reactivity includes diastolic blood pressure changes.

12. The method of claim 1, wherein the measured vascular reactivity includes systolic and diastolic blood pressure changes.

13. The method of claim 1, wherein the vascular reactivity is measured over a period of time of at least 1 minute.

14. The method of claim 1, wherein the vascular reactivity is measured over a period of time of at least 10 minutes.

15. The method of claim 1, wherein the vascular reactivity is measured over a period of time of at least 30 minutes.

16. The method of claim 1, wherein the vascular reactivity is measured continuously.

17. The method of claim 16, wherein the vascular reactivity is measured for at least 10 minutes.

18. The method of claim 1, wherein the skin temperature and/or heat flux are measured during a stress test conducted on the patient.

19. The method of claim 18, wherein the stress test is an adenosine stress test.

20. A method of performing a stress test on a patient, the method comprising:
conducting the stress test; and
measuring vascular reactivity of the patient by measuring skin temperature and/or heat flux from the patient during at least a portion of the stress test.

21. The method of claim 20, wherein the stress test is an adenosine stress test.
